(19) United States
(12) Patent Application Publication
(10) Pub. No.: US 2026/0151610 A1
(43) Pub. Date: Jun. 4, 2026

(54) PROTECTIVE SLEEVE DEVICE AND METHOD

(71) Applicant: Thomas Jefferson University, Philadelphia, PA (US)

(72) Inventors: Paarth Jain, Burke, VA (US); Albert Andy Huang, Tustin, CA (US); Haley Olivia DeMartin, Philadelphia, PA (US); Ethan Locke White, Hummelstown, PA (US); Julia Faye Ponzek, Collegeville, PA (US)

(21) Appl. No.: 19/122,500

(22) PCT Filed: Oct. 18, 2023
(86) PCT No.: PCT/US2023/077166

§ 371 Date: Apr. 18, 2025

Related U.S. Application Data
(60) Provisional application No. 63/379,986, filed on Oct. 18, 2022.

(51) Int. Cl. A61M 39/02

(52) U.S. Cl. CPC A61M 39/0247; A61M 2039/0258; A61M 2039/0288

(57) ABSTRACT

A protective sleeve device for positioning on a limb of a subject, having a sleeve having a proximal end and a distal end, and configured to fit over a limb of a subject, an adjustable cuff positioned at each of the proximal and distal ends of the sleeve; and an adhesive ridge, wherein the adhesive ridge is configured to prevent the proximal cuff from sliding distally down the subject's limb when the adhesive ridge is positioned on the subject's limb under the sleeve and distal to the proximal cuff.

### CROSS-REFERENCE TO RELATED APPLICATIONS

This application claims priority to U.S. Provisional Application No. 63/379,986 filed on Oct. 18, 2022, incorporated herein by reference in its entirety.

### BACKGROUND OF THE INVENTION

IVs are administered frequently in healthcare. In some situations, patients may tamper with the IV attached to their arm. Additionally, the sight of an IV may be unsettling to a patient.

Currently, there are few solutions for preventing a patient from tampering with an IV, while also concealing the IV from view. Existing solutions use healthcare supplies not specifically designed for covering a peripheral IV site or proximal IV line. These solutions include various combinations of medical tape, gauze, compression sleeves, bandages and transparent adhesive pads. Typical healthcare supplies offer inadequate protection and coverage for the IV site and IV line.

A protective sleeve positioned on a limb of a subject and covering an IV has the potential to address many shortcomings found in existing solutions. An example may be an elderly patient prone to disorientation making the individual more likely to pull out an IV. A protective sleeve may prevent the IV from pulling out (or being pulled out) by the subject. Securing the IV during administration may improve the health outcomes of adolescents, elderly, mental health patients, and high-risk individuals more likely to tamper with an IV. Additionally, concealing the IV line and IV site from patient view may increase patient comfort and improve the quality of care.

Thus, there is the need in the art for a protective sleeve for positioning on a limb of a subject that may secure and conceal an IV. The present invention meets this need.

### SUMMARY OF THE INVENTION

In some aspects, a protective sleeve device for positioning on a limb of a subject, having a sleeve having a proximal end and a distal end, and configured to fit over a limb of a subject, an adjustable cuff positioned at each of the proximal and distal ends of the sleeve; and an adhesive ridge, wherein the adhesive ridge is configured to prevent the proximal cuff from sliding distally down the subject's limb when the adhesive ridge is positioned on the subject's limb under the sleeve and distal to the proximal cuff.

In some embodiments, the adhesive ridge comprises a base having a top and bottom surface, and at least one bracket extending up from the top surface. In some embodiments, the bottom surface comprises an adhesive material. In some embodiments, the at least one bracket forms a flange perpendicular to the top surface of the base. In some embodiments, the flange includes a recessed region between the bracket and the top surface of the base.

In some embodiments, the sleeve is a loose fabric material. In some embodiments, the sleeve comprises nylon and spandex. In some embodiments, the adjustable cuff further comprises hook and loop material. In some embodiments, the adjustable cuffs also have a hook backing. In some embodiments, the adhesive ridge comprises at least one thermoplastic material.

In some embodiments, the device further comprises at least one buckle. In some embodiments, the at least one buckle engages with the adjustable cuff to secure the protective sleeve device to the subject. In some embodiments, the buckle comprises a thermoplastic material. In some embodiments, the buckle comprises a rigid material.

In some aspects, a method of securing a protective covering to a limb of subject having the steps of positioning a protective covering comprising a sleeve and adjustable cuff on a limb of subject, such that the sleeve covers a treatment site on the subject's limb, positioning at least one adhesive ridge having a bracket on the limb of the subject, such that the adhesive ridge is under the sleeve and between the treatment site and the adjustable cuff, and securing the sleeve to the limb via the adjustable cuff. In some embodiments, the method also has the step of providing a buckle for engaging the adjustable cuff.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an exemplary embodiment of a protective sleeve device according to aspects of the present invention.

FIG. 2 depicts an exemplary embodiment of a protective sleeve device according to aspects of the present invention.

FIG. 3A depicts an exemplary embodiment of a buckle for a protective sleeve device according to aspects of the present invention. FIG. 3B depicts an exemplary embodiment of a buckle with an exemplary embodiment of a protective sleeve device according to aspects of the present invention. FIG. 3B depicts an exemplary embodiment of a buckle with an exemplary embodiment of a protective sleeve device according to aspects of the present invention.

FIG. 4A depicts an exemplary embodiment of an adhesive ridge for a protective sleeve device according to aspects of the present invention. FIG. 4B and FIG. 4C depict an exemplary embodiment of an adhesive for a protective sleeve device according to aspects of the present invention. FIG. 4D depicts an exemplary embodiment of an adhesive ridge for a protective sleeve device according to aspects of the present invention.

FIG. 5 is a flowchart of an exemplary method for securing a protective covering to the limb of a subject according to aspects of the present invention.

FIG. 6A, FIG. 6B, and FIG. 6C are a series of figures depicting a subject pulling on an exemplary embodiment of a protective sleeve device according to aspects of the present invention.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of the devices and/or methods of the present invention. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Protective Sleeve Device

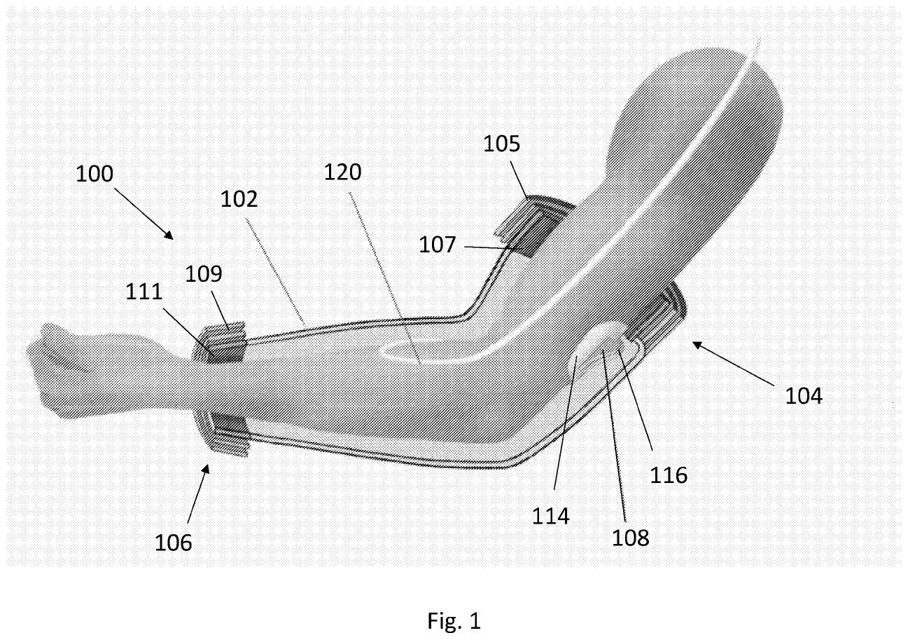

Aspects of the present invention relate to a protective sleeve device that is positioned on a limb of a subject. Now referring to FIG. 1, displayed is an exemplary embodiment of a protective sleeve device positioned on an arm of a subject. In some embodiments, device 100 comprises a sleeve 102 having a proximal cuff 104, a distal cuff 106, and at least one adhesive ridge 108. Also depicted in FIG. 1, separate from device 100, is IV line 120 showing an IV line running from a peripheral IV site to a proximal location on the subject.

Aspects of the present invention relate to a sleeve for a protective sleeve device. In some embodiments, sleeve 102 is a tubular fabric sleeve with a length and at least one diameter that is positioned over a limb of a subject, covering the IV catheter site and the IV line proximal to the site. In some embodiments, device 100 comprises proximal cuff 104, distal cuff 106 with sleeve 102 forming a length therebetween. In some embodiments, device 100 comprises sleeve 102 positioned along the length between proximal cuff 104 and distal cuff 106. In some embodiments, sleeve 102 comprises a fabric material. In some embodiments, sleeve 102 comprises a loose-fitting fabric. In some embodiments, sleeve 102 comprises a breathable, waterproof and/or elastic material. In some embodiments, protective sleeve 102 comprises a 4-way stretch fabric comprising nylon and spandex. In some embodiments, sleeve 102 comprises a plastic material and/or synthetic material. In some embodiments, sleeve 102 comprises one or more holes for accessing a peripheral IV site. In some embodiments, sleeve 102 comprises at least one transparent viewing window with removable cover for viewing a peripheral IV site.

Aspects of the present invention relate to dimensions for an exemplary sleeve for a protective sleeve device. In some embodiments, sleeve 102 has a length of about 8″, 9″, 10″, 11″, 12″, 13″, 14″, 15″, 16″, 17″, 18″, 19″, 20″, or any length therebetween. In some embodiments, sleeve 102 has a length ranging between 1 cm and 50 cm. For example, in some embodiments, protective sleeve has a length of about 11″, or about 28 cm.

Aspects of the present invention relate to the at least one diameter of a sleeve for a protective sleeve device. In some embodiments, sleeve 102 has at least one diameter of about 1″, 2″, 3″, 4″, 5″, 6″, 7″, 8″, 9″, 10″, or any diameter therebetween. In some embodiments, sleeve 102 has a diameter ranging between 1 cm and 25 cm. For example, in some embodiments, protective sleeve has at least one diameter of about 3″, or about 8 cm. In some embodiments, sleeve 102 is in the shape of a frustum (e.g., the frustum of a cone), and comprises a first and second diameter, wherein the first diameter is proximal on sleeve 102 towards proximal cuff 104, and the second diameter is distal on sleeve 102 towards distal cuff 106. For example, in some embodiments, sleeve 102 comprises a first diameter of about 3.26″ and a second diameter of about 2.13″; wherein the second diameter is distal on the sleeve and intended for a human wrist to pass through it.

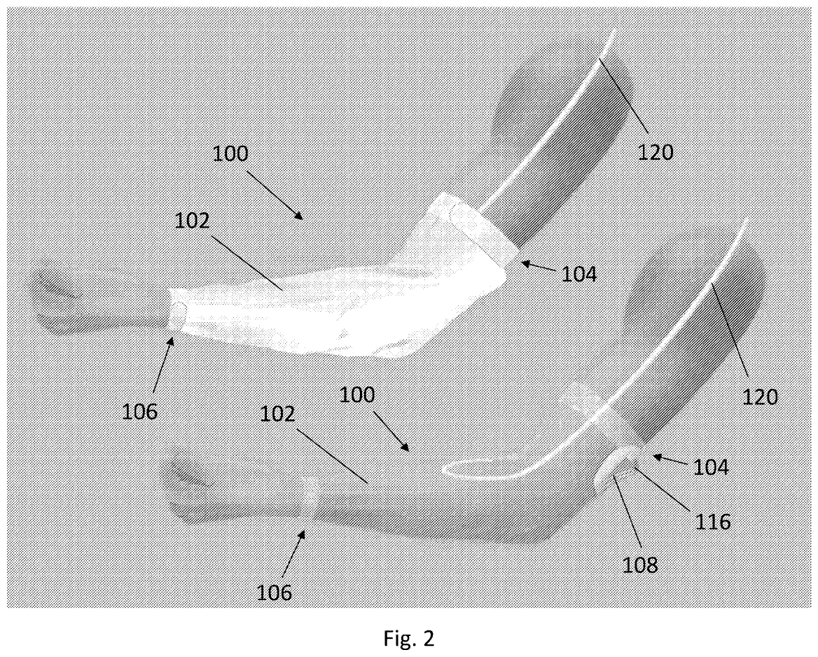

Aspects of the present invention relate to features for securing a protective sleeve to a subject. Now referring to FIG. 1, shown are proximal cuff 104 and distal cuff 106 attached to opposing ends of sleeve 102. In some embodiments, proximal cuff 104 and distal cuff 106 are attached to sleeve 102 by any method as would be known my one of ordinary level of skill in the art. Now referring to FIG. 2, in some embodiments, proximal cuff 104 and distal cuff 106 are securement cuffs for sleeve 102 that may be adjustably tightened via strap, buckle or any other mechanism as would be known by one of ordinary level of skill in the art. In some embodiments, sleeve 102 comprises proximal cuff 104 and distal cuff 106 configured to be tightened to the circumferential size of the subject's limb and secured by hook and loop material and a strap and/or buckle.

In some embodiments, proximal cuff 104 and distal cuff 106 comprise one or more engageable surfaces wherein one fabric may fold and/or interface with another fabric in order to adjust, tighten and/or secure the protective sleeve on the subject. In some embodiments, proximal cuff 104 comprises hook and loop 105 and hook backing 107 to secure the proximal cuff. In some embodiments, distal cuff 106 comprises hook and loop 109 and hook backing 111 to secure the distal cuff. In some embodiments, hook and loop 105 and/or hook and loop 109 comprise a fabric with two sides wherein the first side is hook, and the second side is loop. In some embodiments, hook backing 107 and/or hook backing 111 comprise a fabric with two sides wherein both sides comprise a hook material. In some embodiments, hook and loop 105 and hook backing 107 engage and may be attached together to secure the proximal cuff, and hook and loop 109 and hook backing 111 engage and may be attached together to secure the distal cuff. In some embodiments, hook and loop 105, hook backing 107, hook and loop 109, hook backing 111, and sleeve 102 form a single unit. In some embodiments, hook backing 107, hook backing 111 and sleeve 102 form a single unit and are separate from hook and loop 105 and hook and loop 109. In some embodiments, proximal cuff 104 and/or distal cuff 106 comprise one or more channels wherein an IV line may pass through.

Aspects of the present invention relate to materials for proximal cuff 104 and/or distal cuff 106. In some embodiments, proximal cuff 104 and/or distal cuff 106 comprise a fabric material, a flexible material, an elastic material, a waterproof material, a hook and loop material, a loop material, and/or a hook material. In some embodiments, proximal cuff 104 and distal cuff 106 comprise any fabric securement method as would be known by one of ordinary level of skill in the art, including, but not limited to: buttons, snaps, zippers, hook and loop, Velcro, drawstrings, and the like. In some embodiments, proximal cuff 104 and distal cuff 106 comprise a nylon material. In some embodiments, proximal cuff 104 and distal cuff 106 comprises a spandex material. In some embodiment, proximal cuff 104 and distal cuff 106 comprise inelastic material, elastic material, or combinations thereof. In some embodiments, proximal cuff 104 and distal cuff 106 comprise portions of inelastic material attached to portions of elastic material. In some embodiment, proximal cuff 104 and distal cuff 106 comprise portions of inelastic and/or elastic material attached to hook and loop regions. In some embodiments, proximal cuff 104 and distal cuff 106 comprise a cotton material. In some embodiments, portions of proximal cuff 104 and distal cuff 106 are affixed with thread. In some embodiments, portions of proximal cuff 104 and/or distal cuff 106 are affixed with seamless sewing. In some embodiments, proximal cuff 104 and distal cuff 106 comprise material with a hook receptive backing.

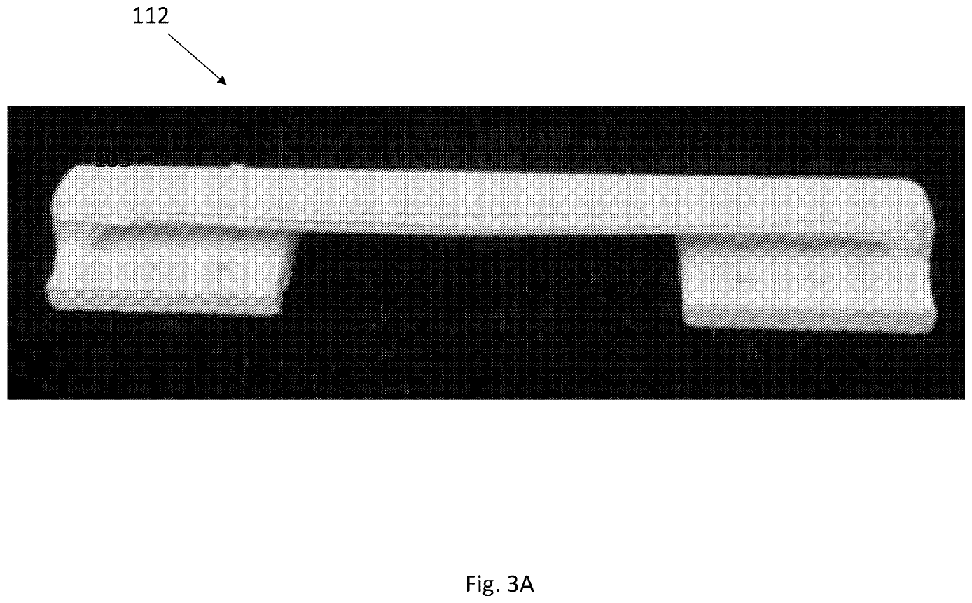

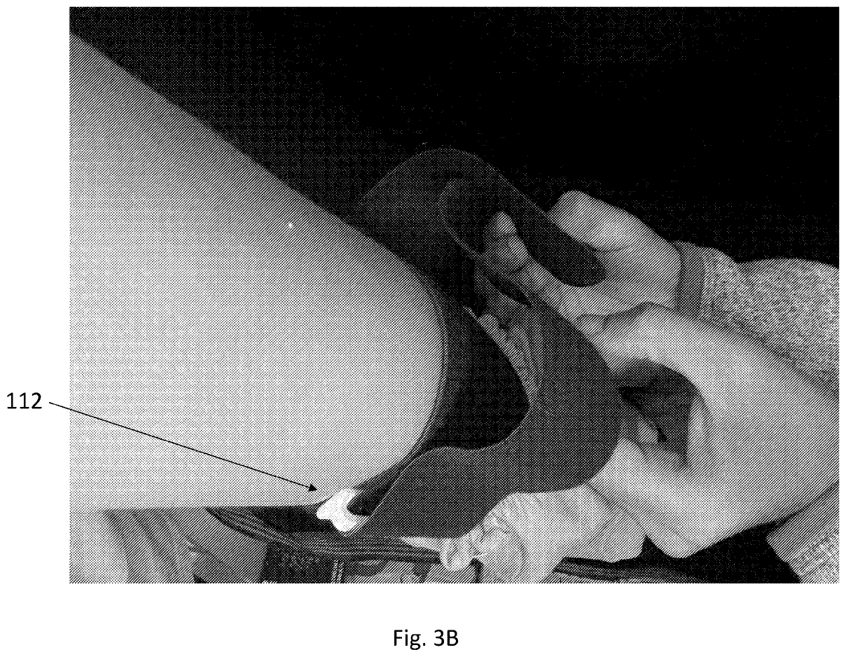

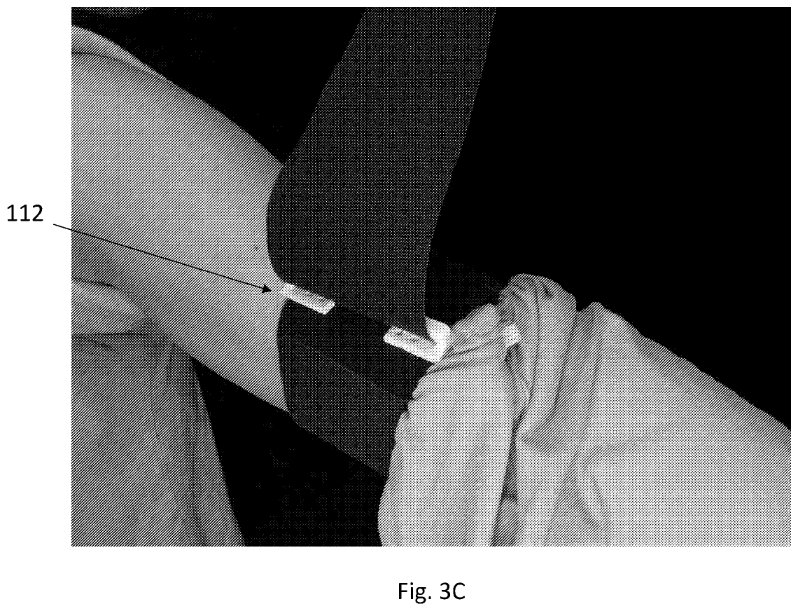

Aspects of the present invention relate to a buckle for a protective sleeve device. Now referring to FIG. 3A, shown is buckle 112 for securing the cuffs of device 100. In some embodiments, device 100 comprises at least one buckle 112 for fastening and/or securing the cuffs of the device. In some embodiments, device 100 comprises at least one buckle wherein hook and loop 105 of proximal cuff 104 and distal cuff 106 may pass through respective buckles on each end and engage with hook backing 107 to secure the cuff. In some embodiments, buckle 112 is formed into proximal cuff 104 and/or distal cuff 106 as one single unit. In some embodiments, buckle 112 is formed as a separate unit from sleeve 102. In some embodiments, hook and loop 105, hook backing 105 and buckle 112 form a rigid fixture on an end of sleeve 102 to prevent tampering by the subject.

Aspects of the present invention relate to the materials for buckle 112 of device 100. In some embodiments, buckle 112 comprises a rigid material. In some embodiments, buckle 112 comprises a plastic material. In some embodiments, buckle 112 comprises a metal material. In some embodiments, buckle 112 comprises a waterproof material. In some embodiments, buckle 112 comprises a 3D printed material. In some embodiments buckle 112 comprises an elastomer. In some embodiments buckle 112 comprises a thermoelastic. In some embodiments buckle 112 comprises a thermoplastic. In some embodiments, buckle 112 comprises thermoplastic urethane, acrylonitrile butadiene styrene, polylactic acid, polyethylene terephthalate glycol, and/or acrylonitrile butadiene styrene.

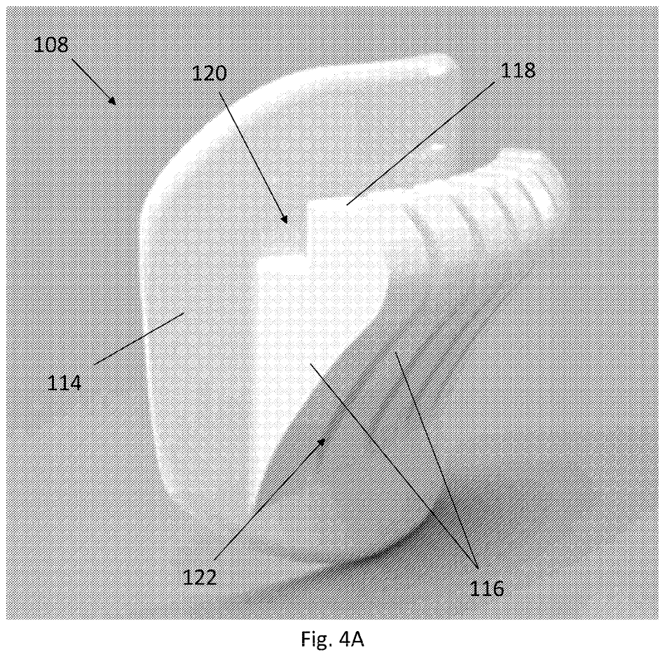

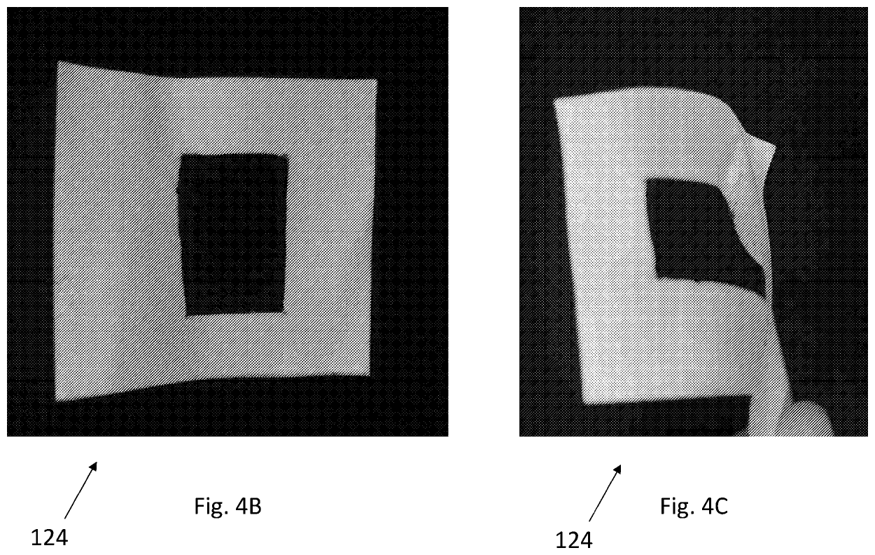

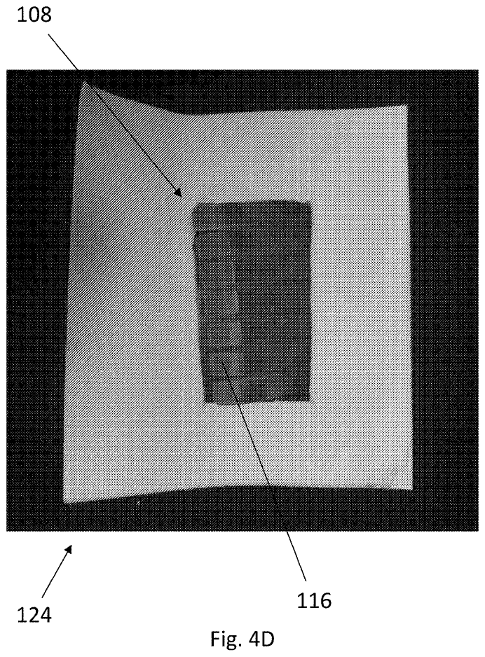

Now referring to FIG. 4A, show is an exemplary embodiment of an adhesive ridge for a protective sleeve device according to aspects of the present invention. The adhesive ridge is designed as a slip guard to prevent sleeve 102 and/or device 100 from moving up or down a limb. In some embodiments, adhesive ridge 108 is configured to prevent movement of sleeve 102, proximal cuff 104, and/or distal cuff 106 on the limb of a subject. In some embodiments, adhesive ridge 108 comprises a base 114 having a top and bottom surface, wherein the bottom surface is configured to contact the skin of a subject. In some embodiments, base 114 comprises a bracket 116 extending up from the top surface of the base, a portion of the bracket forming a prong or extension with a surface 118 and recess 120 for engaging sleeve 102 and proximal cuff 104. In some embodiments, adhesive ridge 108 is flexible such that it is configured to conform and adhere to the surface of the subjects limb. In some embodiments, adhesive ridge 108 is designed to be adhered to the skin over the triceps brachii tendon. It is to be understood that base 112 fixes to skin via any means as would be known by someone of ordinary level of skill in the art.

Aspects of the present invention relate to a bracket on the adhesive ridge for engaging the protective sleeve device. In some embodiments, adhesive ridge 108 comprises at least one bracket 116 with extensions that protrude outward from the top surface of base 114 and form a flange perpendicular to base 114. In some embodiments, adhesive ridge 108 can have a single bracket 116, or multiple brackets 116 spaced apart. In some embodiment, bracket 116 comprises at least one surface 118 that is significantly flat such that objects can catch on it. In some embodiments, adhesive ridge 108 comprises a recess 120 disposed between bracket 116 and base 114. In some embodiments, top surface 118 and recess 120 engage sleeve 102 and/or proximal cuff 104 to prevent movement of the sleeve on the limb of the subject. In some embodiments, adhesive ridge 108 comprises brackets 116 spaced apart in a way such that base 112 can properly flex and conform to the user's limb, while still providing multiple surface 118 and recess 120 along the top surface of base 112. In some embodiments, adhesive ridge comprises at least one gap 122 between each bracket 116 running down to the top surface of base 112 that further enables proper flex of base 112 when conforming to the subjects limb.

Aspects of the present invention relate to features for securing a protective sleeve device to the limb of a subject. Again referring to FIG. 1, in some embodiments, proximal cuff 104 and distal cuff 106 comprise adjustable cuffs wherein hook and loop 105, hook backing 107, hook and loop 109, and hook backing 111 are adjusted to fit the subject. In another example, in some embodiments, portions of sleeve 102 and proximal cuff 104 engage with bracket 116 of adhesive ridge 108 such that protective sleeve 102 and proximal cuff 104 are kept stationary on the limb of the subject. In some embodiments, proximal cuff 104 when tugged engages surface 118 and falls into recess 120 such that the cuff cannot slide down past it. In some embodiments, adhesive ridge 108 is configured to prevent proximal cuff 104 from sliding distally down the subject's limb when adhesive ridge 108 is positioned on the subject's limb under sleeve 102 and distal to proximal cuff 104. It should be appreciated that both proximal cuff 104 and distal cuff 106 each may comprise an adhesive ridge 108, configured to keep the cuff stationary on the limb of the subject.

Aspects of the present invention relate to materials for an adhesive ridge for a protective sleeve device. It should be appreciated that adhesive ridge 108 may also comprise one or more adhesives on the bottom surface of base 114, configured to fixedly and removably adhere adhesive ridge 108 to the limb of a subject.

In some embodiments, adhesive ridge 108 comprises a thermoplastic material. In some embodiments, adhesive ridge 108 comprises a 3D printed material. In some embodiments, ridge 108 comprises a thermoplastic elastomer. In some embodiments, ridge 108 comprises an elastomer. In some embodiments, ridge 108 comprises a thermoelastic. In some embodiments, ridge 108 comprises thermoplastic urethane, acrylonitrile butadiene styrene, polylactic acid, polyethylene terephthalate glycol, and/or acrylonitrile butadiene styrene.

Aspects of the present invention relate to at least one adhesive for adhesive ridge 108. Now referring to FIGS. 4B and 4C, in some embodiments, adhesive ridge 108 comprises adhesive 124 configured to adhere to the skin of a subject. In some embodiments, adhesive 124 comprises a biocompatible adhesive, commercial adhesive, medical adhesive, adhesive layer, adhesive surface, adhesive pad and/or adhesive skirt. In some embodiments, adhesive 124 is configured as a skirt on adhesive ride 108 to hold the ridge in place on the subject's skin.

Now referring to FIG. 4D, depicted is an exemplary adhesive 124 and adhesive ridge 108 according to aspects of the present invention. In one example, adhesive ridge 108 is attached with commercial 3M 2480 medical adhesive. In some embodiments, adhesive 124 comprises a cut out and/or cut through area to allow brackets 116 of adhesive ridge 108 to pass through. In some embodiments, base 114 is configured as a brim where adhesive 124 is configured as a skirt to hold adhesive ridge 108 stationary on a subject's skin.

In some embodiments, adhesive 124 is square in shape. In some embodiments, adhesive 124 is circular in shape. In some embodiments, adhesive 124 is rectangular in shape. In some embodiments, adhesive 124 is oblong in shape. In some embodiments, adhesive 124 has a length and a width. In some embodiments, adhesive 124 has a length of about 4 cm, 5 cm, 6 cm, 7 cm, 8 cm, 9 cm, 10 cm, 11 cm, 12 cm, 13 cm, 14 cm, or about 15 cm. In some embodiments, adhesive 124 has a width of about 4 cm, 5 cm, 6 cm, 7 cm, 8 cm, 9 cm, 10 cm, 11 cm, 12 cm, 13 cm, 14 cm, or about 15 cm.

In some embodiments, adhesive 124 comprises a cut-out interior region with a length and a width to allow brackets 116 of adhesive ridge 108 to pass through. In some embodiments, adhesive 124 comprises a cut-out region with a length of about 1 cm, 2 cm, 3 cm, 4 cm, 5 cm, 6 cm, 7 cm, 8 cm, 9 cm or about 10 cm. In some embodiments, adhesive 124 comprises a cut-out region with a width of about 1 cm, 2 cm, 3 cm, 4 cm, 5 cm, 6 cm, 7 cm, 8 cm, 9 cm or about 10 cm.

### Method of Use

Aspects of the present invention relate to exemplary methods of use for a protective sleeve device. In some embodiments, device 100 is used to cover and/or protect treatment sites on a subject. In some embodiments, device 100 is used to cover and/or protect peripheral IV sites on a subject. In some embodiments, device 100 is used to cover treatment sites on a subject including but not limited to: peripheral IV sites, IV lines, IV ports, wounds bandages, wound dressings, biometric sensors, biometric monitors, and the like. It should be understood that device 100 may be used on any subject receiving an IV. In some embodiments, device 100 is used over the leg. In some embodiments, device 100 is used over the arm. In some embodiments, device 100 is used on non-human subjects.

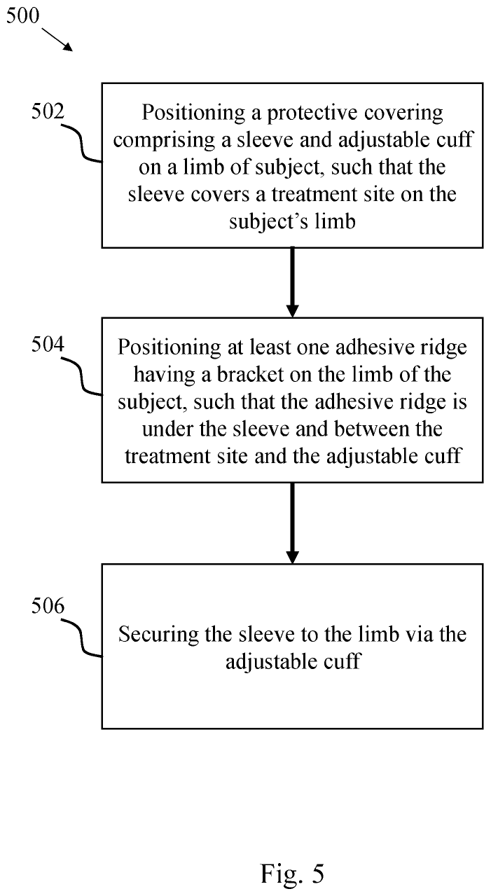

Aspects of the present invention relate to a method for covering and/or protecting a site of treatment. Now referring to FIG. 5, an exemplary method 500 of securing a protective covering to a limb of subject is depicted. Method 500 comprises the steps of: 502 positioning a protective covering comprising a sleeve and adjustable cuff on a limb of subject, such that the sleeve covers a treatment site on the subject's limb, 504 positioning at least one adhesive ridge having a bracket on the limb of the subject, such that the adhesive ridge is under the sleeve and between the treatment site and the adjustable cuff, and, 506 securing the sleeve to the limb via the adjustable cuff.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Pull-Test

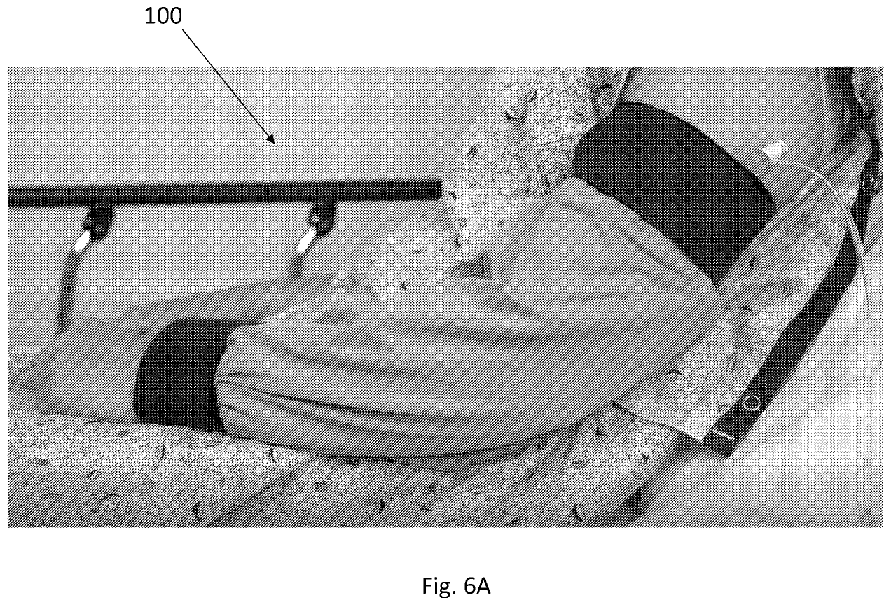

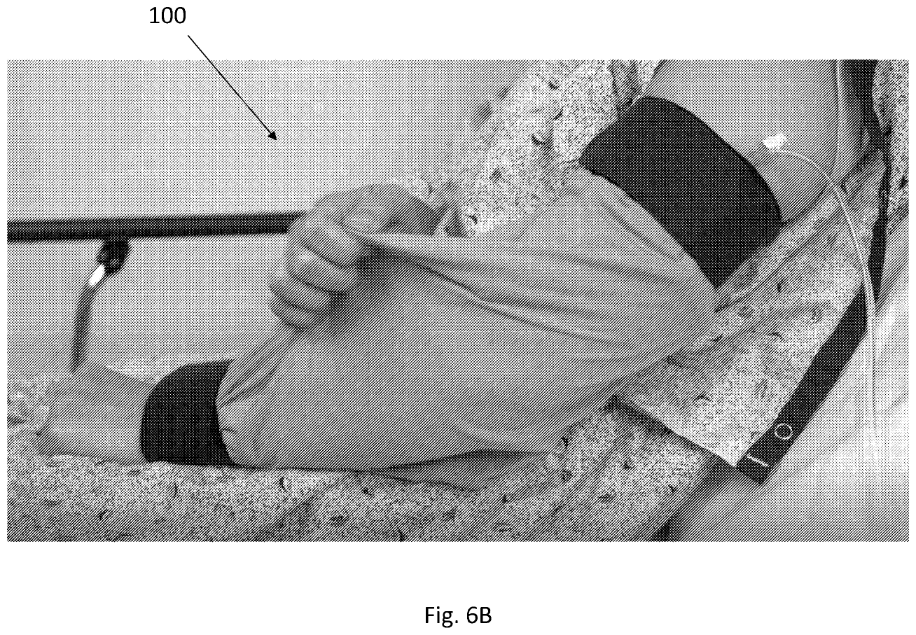

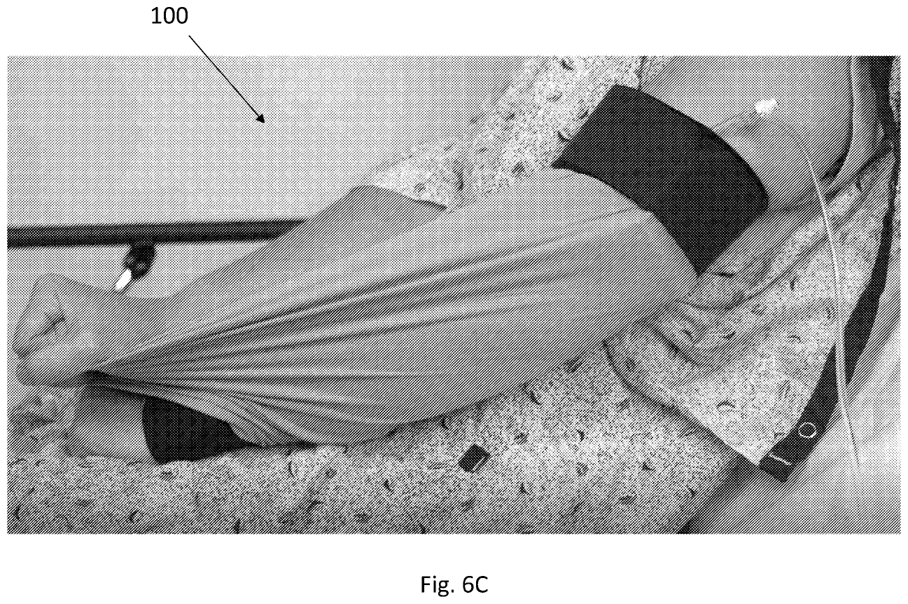

An exemplary protective sleeve device was positioned over the arm of a human subject. The protective sleeve device was tested on the arms of subjects with bicep diameters×arm lengths of: 11 inches×22 inches, 11 inches×19.5 inches, 11.5 inches×20.5 inches, 11.5 inches×21.8 inches. Now referring to FIGS. 6A, 6B, and 6C, shown are a series of figures depicting a subject pulling on a sleeve of an exemplary protective sleeve device. It was show through testing that a protective sleeve device successfully stays on, conceals the IV site, and is able to resist pulling forces. As is shown in FIG. 6C, the sleeve and proximal cuff of the protective sleeve device engage with the adhesive ridge and prevent the distal movement of the protective sleeve device on the arm of the subject. The IV site line are successfully secured, concealed and protected by the protective sleeve device.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A protective sleeve device for positioning on a limb of a subject, comprising:
a sleeve having a proximal end and a distal end, and configured to fit over a limb of a subject;
an adjustable cuff positioned at each of the proximal and distal ends of the sleeve; and
an adhesive ridge;
wherein the adhesive ridge is configured to prevent the proximal cuff from sliding distally down the subject's limb when the adhesive ridge is positioned on the subject's limb under the sleeve and distal to the proximal cuff.

2. The device of claim 1, wherein the adhesive ridge comprises a base having a top and bottom surface, and at least one bracket extending up from the top surface.

3. The device of claim 2, wherein the bottom surface comprises an adhesive material.

4. The device of claim 2, wherein the at least one bracket forms a flange perpendicular to the top surface of the base.

5. The device of claim 4, wherein the flange includes a recessed region between the bracket and the top surface of the base

6. The device of claim 1, wherein the sleeve is a loose fabric material.

7. The device of claim 1, wherein the sleeve comprises nylon and spandex.

8. The device of claim 1, wherein the adjustable cuff further comprises hook and loop material.

9. The device of claim 1, wherein the adjustable cuffs further comprise a hook backing.

10. The device of claim 1, wherein the adhesive ridge comprises at least one thermoplastic material.

11. The device of claim 1, further comprising at least one buckle.

12. The device of claim 11, wherein the at least one buckle engages with the adjustable cuff to secure the protective sleeve device to the subject.

13. The device of claim 12, wherein the buckle comprises a thermoplastic material.

14. The device of claim 12, wherein the buckle comprises a rigid material.

15. The device of claim 1, wherein the adhesive ridge comprises an adhesive skirt.

16. The device of claim 15, wherein the adhesive skirt comprises a medical adhesive with a cut out area to allow portions of the adhesive ridge to pass through.

17. A method of securing a protective covering to a limb of subject comprising the steps of:
positioning a protective covering comprising a sleeve and adjustable cuff on a limb of subject, such that the sleeve covers a treatment site on the subject's limb;
positioning at least one adhesive ridge having a bracket on the limb of the subject, such that the adhesive ridge is under the sleeve and between the treatment site and the adjustable cuff; and
securing the sleeve to the limb via the adjustable cuff.

18. The method of claim 17, further comprising the step of providing a buckle for engaging the adjustable cuff.
